(19) United States
(12) Patent Application Publication
(10) Pub. No.: US 2026/0335382 A1
(43) Pub. Date: Oct. 29, 2026

(54) LACTIC ACID BACTERIUM AND METHOD FOR PREVENTING OR MINIMIZING EXERCISE-INDUCED MUSCLE DAMAGE

(71) Applicant: BENED BIOMEDICAL CO., LTD., Taipei City, Taiwan (TW)

(72) Inventors: Ying-Chieh TSAI, Taipei City (CN); Chi-Chang HUANG, Taipei City (CN); Li-Hao CHENG, Taipei City (CN); Chien-Chen WU, Taipei City (CN); Chih-Chieh HSU, Taipei City (CN)

(21) Appl. No.: 19/124,138

(22) PCT Filed: Oct. 24, 2022
(86) PCT No.: PCT/CN2022/126948

§ 371 Date: Apr. 24, 2025

(51) Int. Cl. A61K 35/747; A61K 35/00; A61P 21/00

(52) U.S. Cl. CPC A61K 35/747; A61P 21/00; A61K 2035/115

(57) ABSTRACT

Provided is a composition for use in preventing or minimizing exercise-induced muscle damage (EIMD) in a subject in need thereof. The composition includes Lactobacillus paracasei PS23 and a carrier thereof. Also provided is a method of preventing or minimizing EIMD by administrating the composition.

### TECHNICAL FIELD

The present disclosure relates to compositions comprising probiotics, in particular, to compositions and methods for preventing or minimizing exercise-induced muscle damage.

### BACKGROUND

Exercise-induced muscle damage (EIMD) occurs after strenuous or prolonged exercise, especially when eccentric muscle contractions are involved^{[1]}, and is characterized by delayed onset muscle soreness (DOMS), structural damage, changes in biochemical parameters and acute local inflammation, such as elevated creatine kinase (CK), lactate dehydrogenase (LDH) and myoglobin (Mb), and elevated inflammatory and oxidative markers (e.g., C-reactive protein (CRP) and malondialdehyde (MDA))^{[2-4]}. These symptoms persist for days and increase if the exercise continues, and are accompanied by a decline in muscle strength and performance, leading to fatigue, which makes it difficult to maintain exercise levels at the desired intensity and thus may negatively impact performance in athletes and amateurs^{[5]}.

For EIMD in professional athletes, mild or moderate DOMS is induced within 2 to 3 days of athletic training. However, for the untrained population, EIMD can lead to severe DOMS and loss of muscle function, such that a longer recovery period is required to fully repair the muscle^{[6]}. Therefore, in addition to an appropriate training program, different strategies must be taken to delay or reduce muscle fatigue, as well as to improve fitness of training, while promoting rapid recovery from high-intensity training or consecutive high-intensity competitions.

Current therapies for muscle repair or regeneration include massage, ultrasound, physical therapy, hyperbaric oxygen delivery, and drug treatments. These therapies may provide some beneficial outcomes, but are far from ideal in terms of, e.g., effectiveness and efficiency.

Therefore, there exists an unmet need to provide for an efficient method that can prevent or reduce exercise-related muscle damages and fatigue, as well as protect a subject from other unfavorable effects resulted from exercise, thereby improving exercise performance.

### SUMMARY

In the present disclosure, a lactic acid bacterium has been identified to be effective in protecting a subject for exercise by, e.g., reducing muscle damage, reducing inflammation, and assisting recovery from fatigue, thereby improving exercise performance in the subject.

In at least one embodiment of the present disclosure, the lactic acid bacterium is Lactobacillus paracasei PS23 (also referred herein to as L. paracasei PS23 or PS23) deposited under DSMZ Accession No. 32322.

The present disclosure provides a composition for use in preventing or minimizing EIMD in a subject in need thereof, wherein the composition comprises an effective amount of a lactic acid bacterium and a carrier thereof. In some embodiments, the lactic acid bacterium is L. paracasei PS23.

In at least one embodiment of the present disclosure, the lactic acid bacterium in the composition is in a form selected from the group consisting of a live strain, a heat-killed strain, a culture, a lysate, an extract, and any combination thereof. In some embodiments, the lactic acid bacterium is heat-killed PS23.

In at least one embodiment of the present disclosure, the composition is administered to the subject at an amount of at least 1×10⁶ colony-forming unit (CFU) of the lactic acid bacterium per day. For example, the effective amount of PS23 is at least 1×10⁶ CFU, at least 1×10⁷ CFU, at least 1×10⁸ CFU, at least 1×10⁹ CFU, at least 1×10¹⁰ CFU or at least 1×10¹¹ CFU, including 5×10⁶ CFU, 5×10⁷ CFU, 5×10⁸ CFU, 2×10⁹ CFU, 3×10⁹ CFU, 4×10⁹ CFU, 5×10⁹ CFU, 6×10⁹ CFU, 7×10⁹ CFU, 8×10⁹ CFU, 9×10⁹ CFU, 2×10¹⁰ CFU, 3×10¹⁰ CFU, 4×10¹⁰ CFU, 5×10¹⁰ CFU, 6×10¹⁰ CFU, 7×10¹⁰ CFU, 8×10¹⁰ CFU, 9×10¹⁰ CFU, 2×10¹¹ CFU, 3×10¹¹ CFU, 4×10¹¹ CFU, 5×10¹¹ CFU, 6×10¹¹ CFU, 7×10¹¹ CFU, 8×10¹¹ CFU, and 9×10¹¹ CFU, but not limited thereto.

In at least one embodiment of the present disclosure, preventing or minimizing EIMD comprises at least one of enhancing muscle strength, increasing muscle endurance, delaying or reducing muscle fatigue, accelerating muscle recovery from EIMD, and preventing or attenuating DOMS.

In at least one embodiment of the present disclosure, the composition is for use in further enhancing physical capacity, improving exercise performance, reducing inflammation, and/or reducing oxidation stress in the subject.

The present disclosure also provides a method of preventing or minimizing EIMD in a subject in need thereof, comprising administering to the subject a composition comprising an effective amount of PS23 and a carrier thereof.

In at least one embodiment of the present disclosure, the method further comprises enhancing physical capacity, improving exercise performance, reducing inflammation, and/or reducing oxidation stress in the subject.

In at least one embodiment of the present disclosure, the composition modulates a serum level of at least one biomarker associated with muscle damage, oxidative stress, inflammation, or anabolic hormones in the subject.

In at least one embodiment of the present disclosure, after the administration of the composition, an increase of at least one of creatine kinase (CK), myoglobin, thiobarbituric acid reactive substances (TBARS), and high-sensitivity C-reactive protein (hs-CRP) resulted from EIMD is suppressed in the subject.

In at least one embodiment of the present disclosure, after the administration of the composition, a reduction of testosterone resulted from EIMD is suppressed in the subject.

In at least one embodiment of the present disclosure, the composition may be a nutritional composition, a food product, or a pharmaceutical composition. In some embodiments, the composition is in a form suitable for oral administration. In some embodiments, the composition is in a form of a concentrate, a paste, a powder, a suspension, a granule, a tablet, a pill, a capsule, a drink, or a dairy product.

In at least one embodiment of the present disclosure, the lactic acid bacterium serves as a sole active ingredient in the composition for preventing or minimizing EIMD. In some embodiments, the lactic acid bacterium may be in combination with an additional active ingredient for preventing or minimizing EIMD.

In at least one embodiment of the present disclosure, the composition may be administered to the subject 1 to 4 times per day or 1 to 4 times per week. In some embodiments, the composition is administered to the subject with a frequency selected from the group consisting of once per week, 2 times per week, 3 times per week, 4 times per week, once every three days, 2 times every three days, once every two days, once per day, 2 times per day, 3 times per day, and 4 times per day.

In at least one embodiment of the present disclosure, the composition is administered to the subject for at least one week. For example, the composition is administered to the subject for at least one week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks.

The composition provided in the present disclosure is effective in reducing inflammation and oxidative stress, as well as improving exercise performance and fighting fatigue. Therefore, by administering the composition, the method of the present disclosure not only provides the benefits of muscle damage recovery, but also enhances physical performance that includes increasing muscle strength, muscle endurance, and physical capacity.

### BRIEF DESCRIPTION OF THE DRAWINGS

The present disclosure can be more fully understood by reading the following descriptions of the embodiments, with reference made to the accompanying drawings.

FIG. 1 shows the experimental procedure performed in the present disclosure. In the intervention, the “star” indicates exercise-induced muscle damage; the “circles” indicate before/after (pre-EIMD) tests that include body composition, dietary record, blood biochemical, CMJ, IMTP and Wingate; and the “triangles” indicate exercise performance test (CMJ, IMTP, Wingate) and assessments of blood biomarkers of muscle damage, inflammation, oxidative stress and anabolic hormones.

FIGS. 2A to 2C show the effects of 6-week L-PS23 and HK-PS23 supplementation on the changes in rate of force development (RFD) (FIG. 2A), relative peak force (FIG. 2B) and jump height (FIG. 2C), measured by countermovement jump assessment (CMJ) at pre-EIMD and 3 h, 24 h, and 48 h post-EIMD. Data are expressed as mean±SD for n=35 subjects per group. Different superscript letters (a, b) indicate significant difference between the groups at the same time point (p<0.05), and * indicates that each group is significantly different from pre-EIMD at different time points (p<0.05). Placebo: the placebo group; L-PS23: the live L. paracasei PS23 group; HK-PS23: the heat-killed L. paracasei PS23 group.

FIGS. 3A and 3B show the effects of 6-week L-PS23 and HK-PS23 supplementation on the changes in relative peak force (FIG. 3A) and peak rate of force development (RFD) (FIG. 3B), measured by isometric mid-thigh pull (IMTP) test at pre-EIMD and 3 h, 24 b, and 48 h post-EIMD. Data are expressed as mean±SD for n=35 subjects per group. Different superscript letters (a, b, c) indicate significant difference between the groups at the same time point (p<0.05), and * indicates that each group is significantly different from pre-EIMD at different time points (p<0.05). Placebo: the placebo group; L-PS23: the live L. paracasei PS23 group; HK-PS23: the beat-killed L. paracasei PS23 group.

FIGS. 4A to 4C show the effects of 6-week L-PS23 and HK-PS23 supplementation on the changes in relative mean power (FIG. 4A), relative peak power (FIG. 4B) and fatigue index (FIG. 4C), measured by Wingate test at pre-EIMD and 3 h, 24 h, and 48 h post-EIMD. Data are expressed as mean±SD for n=35 subjects per group. Different superscript letters (a, b, c) indicate significant difference between the groups at the same time point (p<0.05), and * indicates that each group is significantly different from pre-EIMD at different time points (p<0.05). Placebo: the placebo group; L-PS23; the live L. paracasei PS23 group; HK-PS23: the heat-killed L. paracasei PS23 group.

FIGS. 5A to 5E show the effects of 6-week L-PS23 and HK-PS23 supplementation on the changes in serum levels of creatine kinase (FIG. 5A), myoglobin (FIG. 5B), thiobarbituric acid reactive substances (TBARS) (FIG. 5C), high-sensitivity C-reactive protein (hs-CRP) (FIG. 5D), and testosterone (FIG. 5E), measured at pre-EIMD and 3 h, 24 h, and 48 h post-EIMD. Data are expressed as mean±SD for n=35 subjects per group. Different superscript letters (a, b, c) indicate significant difference between the groups at the same time point (p<0.05), and * indicates that each group is significantly different from pre-EIMD at different time points (p<0.05). Placebo: the placebo group; L-PS23: the live L. paracasei PS23 group; HK-PS23: the heat-killed L. paracasei PS23 group.

### DETAILED DESCRIPTION OF THE EMBODIMENTS

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its scope, for different aspects and applications.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

As used herein, the term “comprising” or “comprises” is used in reference to compositions, methods, and respective component(s) thereof, which are included in the present disclosure, yet open to the inclusion of unspecified elements or steps, whether essential or not.

As used herein, the term “about” generally means within 10%, 5%, 1%, or 0.5% of a given value or range. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

The present disclosure provides a composition for use in preventing or minimizing EIMD in a subject in need thereof, wherein the composition comprises an effective amount of a lactic acid bacterium and a carrier thereof. In some embodiments, the lactic acid bacterium is L. paracasei PS23, which has been deposited under Budapest Treaty at Leibniz Institute DSMZ-German Collection of Microorganisms and Cell Cultures (Inhoffenstr. 7 B, D-38124 Braunschweig, Germany) and has been given the DSMZ Accession No. DSM 32322 by the International Depositary Authority.

As used herein, the phrase “an effective amount” refers to the amount of an active ingredient (e.g., PS23) that confers a desired effect (e.g., preventing or minimizing EIMD) on the treated subject. Effective doses will vary, as recognized by one of ordinary skill in the art, depending on routes of administration, excipient usage, the possibility of co-usage with other treatment, and the condition to be treated.

In at least one embodiment of the present disclosure, the effective amount of PS23 may be at least 1×10⁶ CFU, at least 1×10⁷ CFU, at least 1×10⁸ CFU, at least 1×10⁹ CFU, at least 1×10¹⁰ CFU or at least 1×10¹¹ CFU. In some embodiments, the effective amount of PS23 may range from about 1×10⁶ CFU to about 1×10¹³ CFU, about 1×10⁶ CFU to about 1×10¹² CFU, about 1×10⁶ CFU to about 1×10¹¹ CFU, about 1×10⁶ CFU to about 1×10¹⁰ CFU, about 1×10⁷ CFU to about 1×10¹³ CFU, about 1×10⁷ CFU to about 1×10¹² CFU, about 1×10⁷ CFU to about 1×10¹¹ CFU, about 1×10⁷ CFU to about 1×10¹⁰ CFU, about 1×10⁸ CFU to about 1×10¹³ CFU, about 1×10⁸ CFU to about 1×10¹² CFU, about 1×10⁸ CFU to about 1×10¹¹ CFU or about 1×10⁸ CFU to about 1×10¹⁰ CFU.

As used herein, the term “preventing” or “prevention” refers to preventive or avoidance measures for a disease or a disorder or symptoms or conditions thereof, which include, but are not limited to, applying or administering one or more active agents to a subject who has not yet been diagnosed as a patient suffering from the disease or the disorder or the symptoms or conditions thereof but may be susceptible or prone to the disease or the disorder or the symptoms or conditions thereof. The purpose of the preventive or avoidance measures is to avoid, prevent, or postpone the occurrence of the disease or the disorder or the symptoms or conditions thereof.

As used herein, the term “subject” means a human or animal. Examples of the subject include, but are not limited to, a rodent, a murine, a monkey, a guinea pig, a dog, a cat, a cow, a sheep, a pig, a horse, a rabbit, and a human. In some embodiments, the subject is a mammal, e.g., a primate such as a human.

As used herein, the term “administering” or “administration” refers to the placement of an active ingredient into a subject by a method or route which results in at least partial localization of the active ingredient at a desired site to produce a desired effect. In some embodiments, the composition of the present disclosure is a nutritional composition, a food product, or a pharmaceutical composition and can be in any form suitable for oral administration. For example, the composition may be in a form of a concentrate, a paste, a powder, a suspension, a granule, a tablet, a pill, a capsule, a drink, or a dairy product.

In at least one embodiment of the present disclosure, the nutritional composition may also include a dietary supplement and/or a functional food. The “dietary supplement” designates a product made from compounds usually used in foodstuffs, but which is in the form of tablets, powder, capsules, potion or any other form usually not associated with aliments, and which has beneficial effects for one's health. The “functional food” is an aliment which also has beneficial effects for one's health. In some embodiments, the dietary supplement and the functional food can have a protective or curative effect against a disease or a disorder or the symptoms or conditions thereof.

As used herein, the term “carrier” refers to compounds, materials, compositions, and/or dosage forms which are suitable for use in contact with the tissues of a subject. The carrier is “acceptable” in the sense that it is compatible with the active ingredient (e.g., PS23) of the composition of the present disclosure (and capable of stabilizing the active ingredient) and not deleterious to the subject to be treated. Examples of carriers include colloidal silicon oxide, cellulose, microcrystalline cellulose, sodium lauryl sulfate, polyethylene glycol (PEG), alkylene glycol, sebacic acid, dimethyl sulfoxide, an alcohol, magnesium stearate, calcium stearate, gelatin, fat, glycerin, dietary fiber, alginate, pectin, carrageenan, amidated pectin, xanthan, gellan, karaya gum, rhamsan, welan, gum ghatti, and gum arabic.

The composition provided in the present disclosure may modulate a serum level of at least one biomarker associated with muscle damage, oxidative stress, inflammation, or anabolic hormones in a subject, thereby effectively reducing inflammation and oxidative stress following exercise, as well as improving exercise performance and fighting fatigue.

As used herein, the term “fatigue” refers to skeletal muscle fatigue and/or weakness. Muscle fatigue can be due to strenuous or repeated physical activity or exercise that has symptoms of fatigue, or affects myofibers and/or muscle function. Muscle fatigue may also be defined as the failure of exercise performance.

In at least one embodiment of the present disclosure, the biomarkers associated with muscle damage, oxidative stress, inflammation, and/or anabolic hormones include creatine kinase, myoglobin, thiobarbituric acid reactive substances (TBARS), high-sensitivity C-reactive protein (hs-CRP), and testosterone, but not limited thereto.

Many examples have been used to illustrate the present disclosure. The examples below should not be taken as a limit to the scope of the present disclosure.

### EXAMPLES

### Materials and Methods

The materials and methods used in the following Examples 1 to 4 are described in detail below. The materials used in the present disclosure but unannotated herein are commercially available.

### (1) Preparation of Probiotic Formulation

L. paracasei PS23 was isolated from healthy human feces and has been deposited under DSMZ Accession No. 32322. For preparing nutritional supplement, the PS23 was inoculated in a culture medium, cultured at 37 °C for 18 hours, harvested by centrifugation, and embedded and lyophilized with protective agents and excipients to form a live PS23 powder with a final concentration of 1×10¹¹ colony-forming unit (CFU) per gram. As to heat-killed L. paracasei PS23, the PS23 was cultured as described above and heat-treated at 80 °C for 1 hour before centrifugation, and then embedded and lyophilized with protective agents and excipients to form a heat-killed PS23 powder with a final concentration of 1×10¹¹ cells per gram. Subsequently, the live and heat-killed L. paracasei PS23 powders were formulated into capsules, wherein the L-PS23 treatment capsule contained 10 billion (1×10¹⁰) CFU of live PS23, and the HK-PS23 treatment capsule contained 10 billion cells of heat-killed PS23.

The placebo capsule contained only microcrystalline cellulose (MCC) and matched to the L-PS23 and HK-PS23 treatment capsule for size, color, and taste.

### (2) Participants

The study described herein was conducted according to the guidelines of the Declaration of Helsinki. Experiments were performed after approval by the Institutional Review Board of Landseed International Hospital (Taoyuan, Taiwan; LSHIRB No. 22-003-A2). The subjects aged 20 to 40 years old were recruited in this study, and anyone with one of the following conditions was excluded: (1) having a history of cardiovascular disease, hypertension, metabolic disease, asthma or cancer; (2) body mass index (BMI) ≥27; (3) having limb impairment, neuromuscular motor impairment, and the like within 6 months and not able to engage in exercise; (4) taken anti-inflammatory, analgesic, or acute and chronic diseases and other drugs in the last month; (5) having conflict of interest with the principal investigator; (6) having the habit of smoking and drinking; (7) using probiotic powder, capsule or lozenge (including yogurt, and other related foods) within two weeks; (8) having food or lactic acid bacteria products allergy; and (9) having undergone hepatobiliary gastrointestinal surgery (except hernia and polypectomy).

A total of 114 subjects from National Taiwan Sport University were included in this study, including 78 males and 36 females. All subjects were randomly assigned to 3 groups (i.e., L-PS23 group, HK-PS23 group, and placebo group) with an equal ratio of males and females in each group (26 males and 12 females/group). All participants signed a written informed consent form before starting the experiment. During the experiment, a small number of subjects in each group withdrew from the experiment due to changes in their physiological conditions, such as being diagnosed with COVID-19 or temporarily unable to cooperate with the experimental schedule.

### (3) Experimental Design

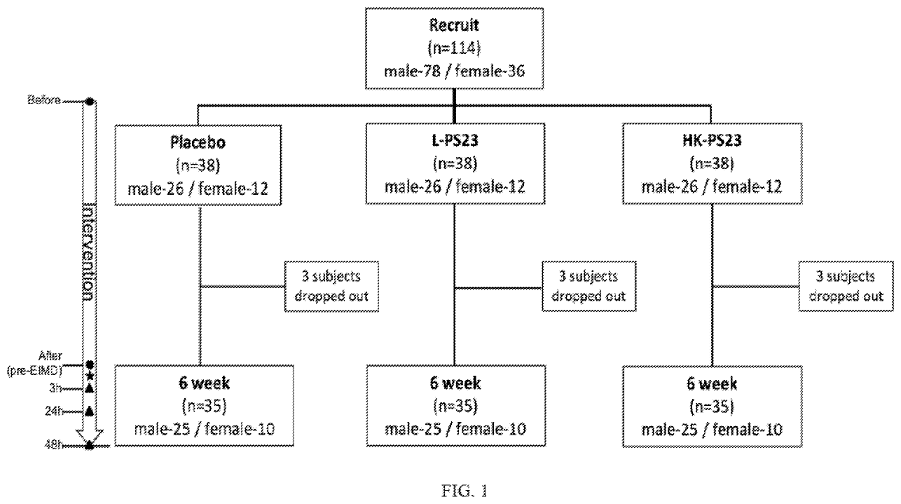

The study described herein used a double-blind design. The experimental procedure was shown in FIG. 1. All subjects were asked to take two capsules per day for 6 weeks, in which the subjects of the L-PS23 group took the L-PS23 capsules (1×10¹⁰ CFU/capsule, 2 capsules/day), the subjects of the HK-PS23 group took the HK-PS23 capsules (1×10¹⁰ cells/capsule, 2 capsules/day), and the subjects of the placebo group took the placebo capsules (2 capsules/day).

Participants visited the laboratory a total of 5 times. Body composition, basal blood biochemical parameters, and exercise performance were measured before placebo, L-PS23 or HK-PS23 supplementation and six weeks after intervention, followed by immediate exercise-induced muscle damage (EIMD). Biomarkers of muscle damage, indicators of inflammation and oxidative stress, and anabolic hormones were measured at 3, 24, and 48 hours after EIMD.

All participants were asked to maintain their daily diet throughout the study period and were asked to record their dietary intake for professional nutritionist to analyze. The basic information and performance of physical parameters of the subjects before the intervention were reported in Table 1 below.

TABLE 1
Participant characteristics and physical fitness test at baseline

| Basic | | | | |
|---|---|---|---|---|
| information | Parameter (unit) | Placebo | L-PS23 | HK-PS23 |
| Physical | Age (years) | 21.6 ± 2.0^{a} | 21.4 ± 1.3^{a} | 21.8 ± 2.5^{a} |
| characteristics | Height (cm) | 172 ± 9^{a} | 172 ± 9^{a} | 171 ± 9^{a} |
| | Weight (kg) | 69.4 ± 12.4^{a} | 69.7 ± 11.0^{a} | 66.4 ± 11.6^{a} |
| | BMI (kg/m²) | 23.2 ± 2.8^{a} | 23.5 ± 2.4^{a} | 22.7 ± 2.6^{a} |
| CMJ | RFD (N/kgxsec) | 8.11 ± 0.95^{a} | 8.03 ± 1.03^{a} | 7.96 ± 1.08^{a} |
| | Relative peak force (N/kg) | 15.11 ± 2.12^{a} | 15.23 ± 2.22^{a} | 15.09 ± 2.55^{a} |
| | Jump height (g/day) | 30.7 ± 6.0^{a} | 31.6 ± 7.6^{a} | 32.0 ± 6.5^{a} |
| IMTP | Relative peak force (N/kg) | 15.57 ± 2.32^{a} | 15.31 ± 3.06^{a} | 15.46 ± 2.51^{a} |
| | Peak RFD (N/sec) | 9244 ± 1777^{a} | 9219 ± 1821^{a} | 9102 ± 1520^{a} |
| Wingate | Relative mean power (W/kg) | 6.17 ± 0.91^{a} | 6.53 ± 0.94^{a} | 6.45 ± 0.97^{a} |
| | Relative peak power (W/kg) | 9.61 ± 1.59^{a} | 9.40 ± 1.76^{a} | 9.38 ± 1.73^{a} |
| | Fatigue index (%) | 48.6 ± 3.9^{a} | 47.8 ± 4.2^{a} | 48.1 ± 4.6^{a} |

Same superscript letters ^{a}indicated no significant difference between the groups, p > 0.05.
BMI: body mass index; CMJ: countermovement jump; IMTP: isometric mid-thigh pull; RFD: rate of force development; Wingate: Wingate anaerobic test.

### (4) Body Composition Measurement

All subjects were asked to fast for at least 8 hours prior to the measurement. During the measurement by an In-Body 570 device (In-body, Seoul, South Korea), the subject stood on the bottom electrode, held the sensing handle with both hands, did not speak or move, and kept the arms open so that the arms were away from the body at a 30-degree angle. Body composition was measured according to the multi-frequency principle with a Bioelectrical Impedance Analyzer (BIA) at 1, 5, 50, 260, 500 and 1000 kHz within 60 seconds.

### (5) Elicitation of Exercise-Induced Muscle Damage (EIMD)

For eliciting EIMD, all subjects were asked to perform 100 maximal vertical jumps. This method involved jumping every 4 seconds for 10 times as a group, for a total of 10 groups. All subjects were asked to bend their knees 90 degrees during each squat, and each subject's maximum jump height was marked as the goal. The researchers encouraged the subjects to try their best to reach the maximum height target with each jump.

### (6) Countermovement Jump Assessment (CMJ)

The CMJ was used to measure the maximum speed, strength, and explosiveness of the lower body. For this test, participants were asked to place their hands on their hips and stand with their feet on a Kistler force-measuring platform (9260AA, Kistler GmbH, Switzerland). Afterwards, participants were instructed to squat until their knees were bent 90 degrees and then immediately jump as high as possible with maximum force. Each participant received 3 replicates. The CMJ data were obtained at designated points, and the instrument was calibrated for each participant's weight. The measured parameters included rate of force development (RED), relative peak force, and jump height, wherein RED is defined as the ability to generate force at a given time during rapid voluntary contractions and is often used to assess blast strength.

### (7) Isometric Mid-Thigh Pull (IMTP) Test

The IMTP test has been shown to be a valid and reliable test of maximal lower extremity strength that is highly correlated with exercise performance and used for changes in explosive power and as a marker of fatigue. For the test, a custom-made IMTP test equipment (IMTP Rack, Kairos Strength, USA) and force plate (Model 9260AA, Kistler, Switzerland) were used. All subjects stood with feet equal-width apart, the bar between the thighs, the torso upright, the spine neutral, and the knees and hips at a 140-degree angle, and then pulled the bar with maximum force for 3 to 5 seconds.

Before the test, subjects should be familiar with the process of IMTP test and exercise to ensure correct force and stable weight frequency. Measurements were repeated at 2-minute intervals for each test to reduce detection errors related to postural changes. The measured parameters included relative peak force and peak RED.

### (8) Wingate Anaerobic Test (WANT)

Participants were instructed to complete a full sprint on an anaerobic power bike (894E, Monark Exercise AB, Sweden) within 30 seconds. The seat height was adjusted individually for each participant, and toe clips prevented feet from slipping off the pedals. The measurement was started after warming up. When the subjects sprinted at 120 revolutions per minute (rpm), a weight set to 7.5% (for example, a 60 kg person would add 4.5 kg of resistance) automatically landed on the bike's friction belt, thereby creating additional resistance. The subjects were asked to continuously sprint at full strength until 30 seconds. After the test, the subjects' fatigue index was recorded by using the rating of perceived exertion. The measured parameters included relative mean power, relative peak power and fatigue index.

### (9) Assessments of Biomarkers of Muscle Damage, Inflammation, Oxidative Stress and Anabolic Hormones

Blood-related indicators are one of the effective indirect methods for assessing muscle damage and inflammation. For example, creatine kinase (CK) is typically elevated after exercise and is frequently used as a biomarker of skeletal muscle tissue damage.

Venous blood was collected from subjects at pre-EIMD (i.e., after the 6-week sample intervention and before the EIMD elicitation), and 3 h, 24 h and 48 h post-EIMD for biomarker assessments, including the biomarkers of muscle damage, inflammation, oxidative stress and anabolic hormones.

The levels of creatine kinase (CK) and high-sensitivity CRP (hs-CRP) were analyzed by autoanalyzer (AU 5820, Beckman Coulter, Inc., CA, USA). The levels of myoglobin and testosterone were analyzed by autoanalyzer (DXI 800, Beckman Coulter, Inc., CA, USA). The thiobarbituric acid reactive substances (TBARS) level was measured by the TBARS assay kit (No. 10009055, Cayman Chemical Company, Ann Arbor, Michigan, USA).

### (10) Serum Biochemical Analysis

The subject's venous blood was collected before and after the 6-week sample intervention, and used for analyzing liver function, renal function, blood lipids and other indicators to evaluate the subjects' metabolic and health status, thereby determining whether they were affected by the intervention. All the subjects were asked for fasting at least 8 hours, and the blood serum was assessed with an autoanalyzer (Hitachi 717, Tokyo, Japan) for the levels of aspartate transaminase (AST), aminotransferase (ALT), total cholesterol (TC), triglyceride (TG), high-density lipid-cholesterol (HDL-C), low-density lipid-cholesterol (LDL-C), blood urea nitrogen (BUN), creatine (CREA), uric acid (UA) and glucose.

### (11) Statistical Analysis

All the data were expressed as mean±SD. Statistical analyses were performed by SAS 9.0 (SAS Inst., Cary, NC, USA). Multi-group comparisons were analyzed by one-way analysis of variance (ANOVA). By IBM SPSS Statistics version 24.1 (IBM, Co., Armonk, NY, USA), the repeated measures ANOVA was used to compare placebo, L-PS23 and HK-PS23 at specific time points during recovery. Paired t-tests with Bonferroni adjustment were used to compare treatment differences with pre-EIMD at each time point. Statistical significance was set at p<0.05.

### Example 1: Effects on Dietary Intake and Body Composition Before and After the 6-Week L-PS23 or HK-PS23 Intervention

Table 2 showed the subjects' daily dietary intake (carbohydrate, protein, and fat) and total calories before and after six weeks of the intervention. The results indicated that there was no significant difference between the three groups, and there was no significant change in the before-after comparison within the group.

TABLE 2
Subject's dietary intake before and after the 6-week L-PS23 or HK-PS23 intervention

| Dietary | Before | | | After | | |
|---|---|---|---|---|---|---|
| Intake | Placebo | L-PS23 | HK-PS23 | Placebo | L-PS23 | HK-PS23 |
| Carbohydrate (g/day) | 214 ± 69^{a} | 213 ± 37^{a} | 208 ± 34^{a} | 214 ± 70^{a} | 215 ± 48^{a} | 213 ± 36^{a} |
| Protein (g/day) | 152 ± 23^{a} | 155 ± 20^{a} | 147 ± 27^{a} | 149 ± 23^{a} | 155 ± 19^{a} | 149 ± 23^{a} |
| Fat (g/day) | 92 ± 5^{a} | 93 ± 6^{a} | 90 ± 5^{a} | 93 ± 5^{a} | 93 ± 9^{a} | 92 ± 6^{a} |
| Total calorie (kcal/day) | 2287 ± 313^{a} | 2304 ± 163^{a} | 2232 ± 153^{a} | 2289 ± 315^{a} | 2319 ± 219^{a} | 2274 ± 194^{a} |

Same superscript letters ^{a} indicated no significant difference between the groups, p > 0.05.

Further, in terms of body composition, the change to the subjects' body composition before and after the intervention was reported in Table 3. It was shown that there were no significant differences in body weight, body mass index (BMI), lean body mass (LBM), and fat body mass (FBM) between the groups before and after six weeks of the intervention.

However, in the placebo group after six weeks of the intervention compared with those before intervention, body weight (p=0.0084), BMI (p=0.0087) and FBM (p=0.0044) were significantly increased, and LBM (p=0.0072) was decreased. In the HK-PS23 group after six weeks of the intervention, body weight (p=0.0036) and BMI (p=0.0028) were decreased as compared with those before intervention. In addition, the body weight and BMI of the HK-PS23 group were significantly reduced as compared with the placebo and L-PS23 groups (p<0.05), and FBM of the HK-PS23 group was also significantly reduced as compared with the placebo group (p=0.0109).

TABLE 3
Subject's body composition before and after the 6-week L-PS23 or HK-PS23 intervention

| | Before | | | After | |
|---|---|---|---|---|---|
| Parameters | Placebo | L-PS23 | HK-PS23 | Placebo | L-PS23 |
| BW (kg) | 69.4 ± 12.4^{a} | 69.7 ± 11.0^{a} | 66.4 ± 11.6^{a} | 70.1 ± 12.9^{a,} * | 70.0 ± 11.0^{a} |
| BMI (kg/m²) | 23.2 ± 2.8^{a} | 23.5 ± 2.4^{a} | 22.7 ± 2.6^{a} | 23.5 ± 3.0^{a,} * | 23.6 ± 2.5^{a} |
| LBM (kg) | 31.8 ± 6.7^{a} | 31.7 ± 6.3^{a} | 30.2 ± 6.2^{a} | 31.5 ± 6.7^{a,} * | 31.7 ± 6.4^{a} |
| FBM (%) | 20.0 ± 7.0^{a} | 20.1 ± 7.9^{a} | 19.3 ± 6.5^{a} | 20.8 ± 6.7^{a,} * | 20.2 ± 8.5^{a} |

| | After | Delta | | |
|---|---|---|---|---|
| Parameters | HK-PS23 | Placebo | L-PS23 | HK-PS23 |
| BW (kg) | 65.6 ± 11.7^{a,} * | 0.7 ± 1.4^{b} | 0.3 ± 2.3^{b} | −0.8 ± 1.6^{a} |
| BMI (kg/m²) | 22.4 ± 2.7^{a,} * | 0.2 ± 0.5^{b} | 0.1 ± 0.8^{b} | −0.3 ± 0.5^{a} |
| LBM (kg) | 30.2 ± 6.0^{a} | −0.3 ± 0.6^{a} | 0.0 ± 0.9^{a} | −0.1 ± 0.6^{a} |
| FBM (%) | 19.1 ± 6.0^{a} | 0.8 ± 1.5^{b} | 0.1 ± 1.4^{ab} | −0.2 ± 1.7^{a} |

Different superscript letters ^{a, b} indicated significant difference between the groups on the same time point, p < 0.05.
*indicated a significant effect after intervention as compared to that before intervention, p < 0.05.
Delta indicated the value of “after intervention” minus “before intervention.”
BW: body weight; BMI: body mass index; LBM: lean body mass; FBM: fat body mass.

### Example 2: Effects on Biochemical Characteristics Before and After 6-Week L-PS23 or HK-PS23 Intervention

Before and after the intervention, blood was collected from all subjects for basic blood parameter analysis to understand the healthy physiological state of the subjects before the intervention, thereby determining whether there were any adverse effects or side effects after the 6-week PS23 intervention.

As shown in Table 4, the results indicated that the liver function indexes (AST and ALT), renal function indexes (BUN, CREA, and UA), blood lipid metabolism indexes (TC, TG, HDL-C, and LDL-C) and blood sugar were all within the normal range, and there was no significant difference between the three groups before or after the intervention. In addition, there were no significant changes in each group after intervention as compared with those before intervention. Also, there were no adverse events related to intervention during the study.

TABLE 4
Subject's blood biochemical parameters before and after the 6-week L-PS23 and HK-PS23 intervention

| | Before | | | After | | |
|---|---|---|---|---|---|---|
| Parameters | Placebo | L-PS23 | HK-PS23 | Placebo | L-PS23 | HK-PS23 |
| AST (U/L) | 21 ± 6^{a} | 22 ± 7^{a} | 21 ± 7^{a} | 21 ± 6^{a} | 22 ± 6^{a} | 121 ± 6^{a} |
| ALT (U/L) | 20 ± 6^{a} | 22 ± 7^{a} | 20 ± 7^{a} | 20 ± 4^{a} | 22 ± 7^{a} | 21 ± 7^{a} |
| TC (mg/dL) | 179 ± 16^{a} | 173 ± 18^{a} | 174 ± 22^{a} | 174 ± 21^{a} | 178 ± 15^{a} | 172 ± 18^{a} |
| TG (mg/dL) | 81 ± 24^{a} | 84 ± 24^{a} | 81 ± 18^{a} | 82 ± 22^{a} | 85 ± 22^{a} | 82 ± 17^{a} |
| HDL-C (mg/dL) | 57.5 ± 9.3^{a} | 56.7 ± 8.5^{a} | 57.5 ± 7.6^{a} | 58.0 ± 9.5^{a} | 56.8 ± 9.1^{a} | 58.2 ± 8.3^{a} |
| LDL-C (mg/dL) | 96.5 ± 17.7^{a} | 98.2 ± 15.2^{a} | 97.1 ± 17.3^{a} | 96.5 ± 18.0^{a} | 97.2 ± 15.2^{a} | 96.0 ± 16.5^{a} |
| BUN (mg/dL) | 13.8 ± 2.5^{a} | 14.9 ± 2.7^{a} | 14.7 ± 3.1^{a} | 13.5 ± 2.9^{a} | 14.5 ± 2.3^{a} | 14.4 ± 2.5^{a} |
| CREA (mg/dL) | 1.10 ± 0.10^{a} | 1.10 ± 0.11^{a} | 1.09 ± 0.12^{a} | 1.06 ± 0.12^{a} | 1.10 ± 0.11^{a} | 1.07 ± 0.10^{a} |
| UA (mg/dL) | 5.7 ± 0.9^{a} | 5.6 ± 0.9^{a} | 5.4 ± 1.0^{a} | 5.5 ± 0.8^{a} | 5.6 ± 1.0^{a} | 5.3 ± 1.1^{a} |
| Glucose (mg/dL) | 86 ± 6^{a} | 84 ± 6^{a} | 84 ± 8^{a} | 84 ± 6^{a} | 84 ± 7^{a} | 84 ± 7^{a} |

Same superscript letters ^{a} indicated no significant difference between the groups, p > 0.05.
AST: aspartate aminotransferase; ALT: alanine transaminase; TC: total cholesterol; TG: triacylglycerol; HDL-C: high-density lipid-cholesterol; LDL-C: low-density lipid-cholesterol; BUN: blood urea nitrogen; CREA: creatinine; UA: uric acid.

### Example 3: Effects of L-PS23 and HK-PS23 Supplementation on Exercise Performance

### (1) Vertical Jump Height

Vertical jumping has been used to evaluate athletes' speed, maximal strength, explosive power, and anaerobic performance^{[7]}, and used as one of the fatigue indicators of muscle damage and decreased muscle strength^{[8]}.

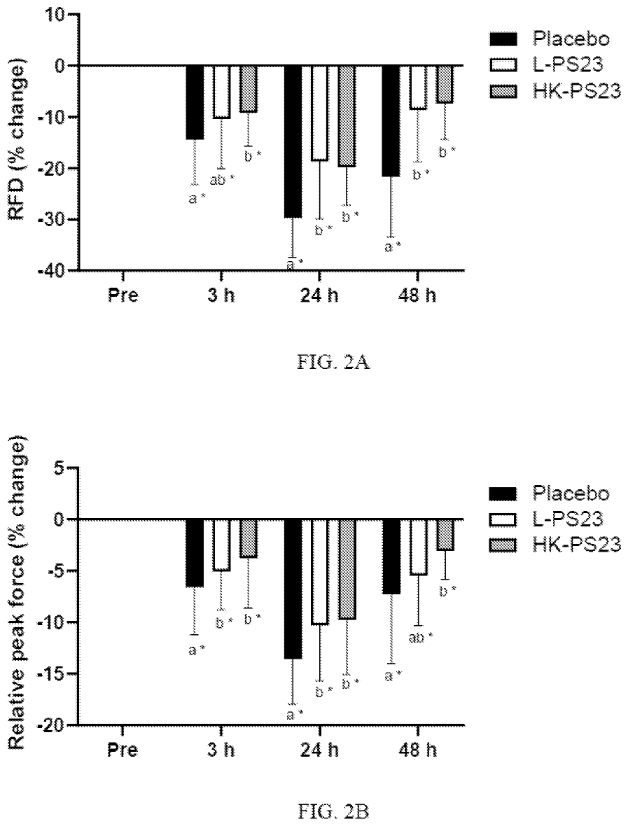

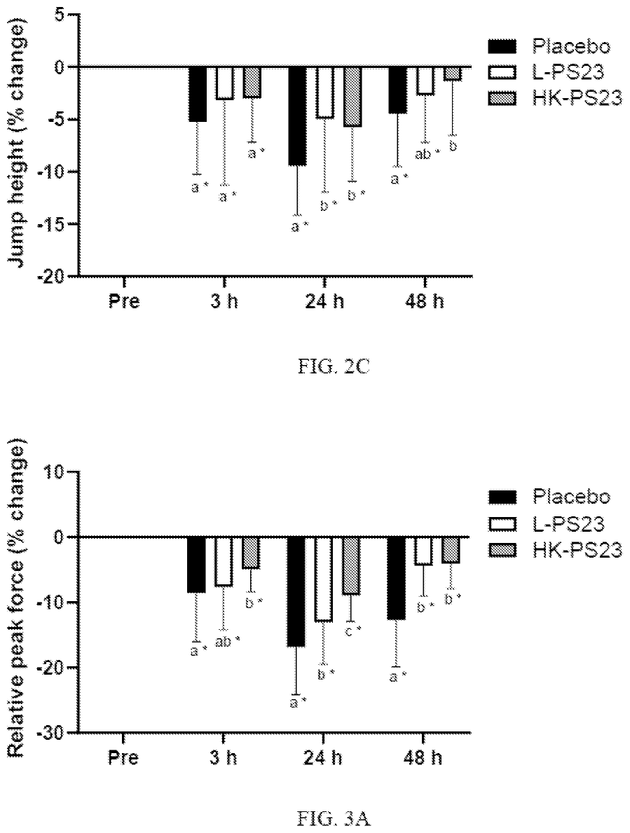

The vertical jump height was measured by countermovement jump assessment (CMJ) at 3 h, 24 h, and 48 h post-EIMD. It was found that the percentage changes in rate of force development (RFD) and relative peak force were significantly lower in all three groups, namely the placebo, L-PS23, and HK-PS23 groups, at 3 h, 24 h, and 48 h post-EIMD as compared with pre-EIMD (p<0.05) (FIGS. 2A and 2B).

Further, from the between-group comparisons, FIG. 2A showed that the L-PS23 or HK-PS23 supplementation group had significantly less RFD loss and faster recovery than the placebo group at 24 and 48 hours after EIMD (p<0.0001), and the HK-PS23 group had significantly less RFD loss than the placebo group at 3 h post-EIMD (p<0.05). In addition, as shown in FIG. 2B, the L-PS23 and HK-PS23 supplementation groups had significantly less relative peak force loss than the placebo group at 3 h and 24 h post-EIMD (p<0.05), and the HK-PS23 group had significantly faster recovery than the placebo group at 48 h post-EIMD (p=0.0008).

As shown in FIG. 2C, the jump height in the L-PS23 and HK-PS23 supplementation groups had significantly less loss than the placebo group at 24 h post-EIMD (p<0.05). Also, the HK-PS23 group had significantly faster recovery than the placebo group at 48 h post-EIMD (p=0.0097) and had no significant difference as compared with pre-EIMD. In addition, these results indicated significant time effects for RFD, relative peak force and jump height (p<0.0001).

### (2) Isotonic Muscle Strength

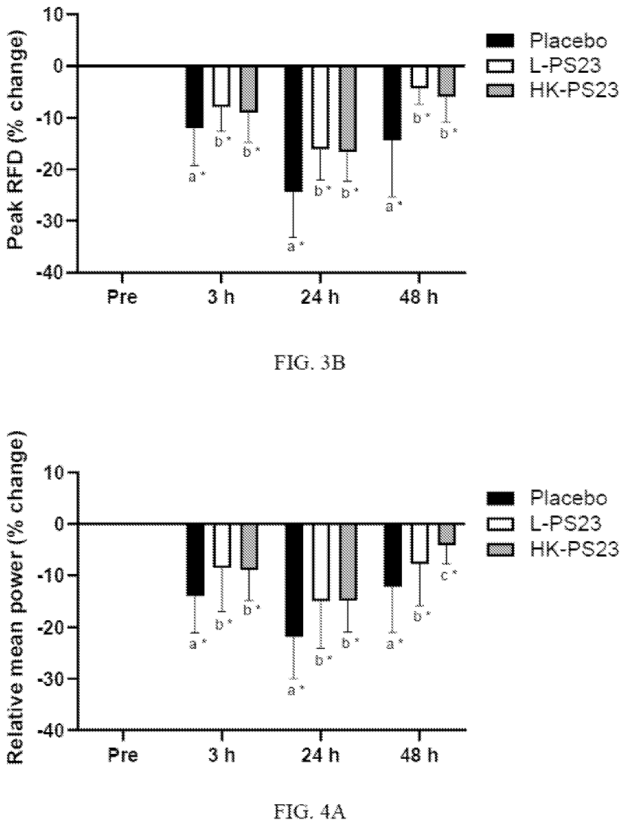

By the isometric mid-thigh pull (IMTP) test at 3 h, 24 h, and 48 h post-EIMD, it was found that the percentage changes in relative peak force and peak RFD were significantly lower in the placebo, L-PS23, and HK-PS23 groups as compared with pre-EIMD (p<0.0001) (FIGS. 3A and 3B). Further, the L-PS23 group had significantly less loss of relative peak force at 24 h and 48 h post-EIMD, and the HK-PS23 group had significantly less loss of relative peak force at 3 h, 24 h and 48 h post-EIMD as compared with the placebo group (p<0.05) (FIG. 3A).

On the other hand, the L-PS23 and HK-PS23 groups had significantly less loss of peak RFD at 3 h and 24 h post-BIMD, and had significantly better recovery at 48 h post-EIMD than those of the placebo group (p<0.05) (FIG. 3B). In addition, these results indicated significant time effects for relative peak force and peak RFD (p<0.0001).

### (3) Anaerobic Exercise Performance

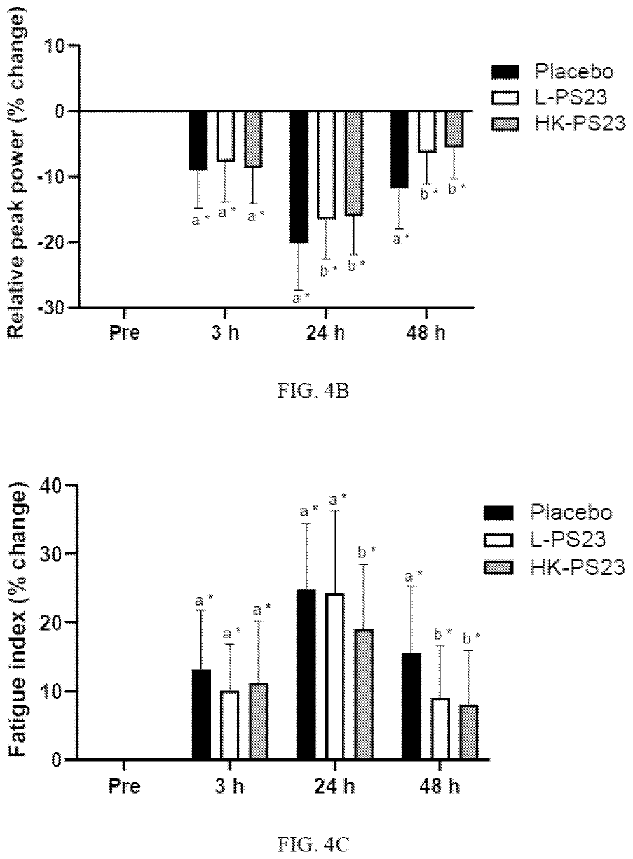

The Wingate anaerobic test was used to evaluate the effects of lower limb muscle strength and anaerobic strength after EIMD. By the Wingate anaerobic test at 3 h, 24 h, and 48 h post-EIMD, it was found that in the placebo, L-PS23, and HK-PS23 groups, the percentage changes in relative mean power and relative peak power were significantly lower, and the percentage change in fatigue index was significantly greater, as compared with pre-EIMD (p<0.0001) (FIGS. 4A to 4C).

Further, the L-PS23 and HK-PS23 groups had significantly less loss of relative mean power than the placebo group at 3 h, 24 h, and 48 h post-EIMD (p<0.05) (FIG. 4A). As shown in FIG. 4B, the L-PS23 and HK-PS23 groups had significantly less loss of relative peak power than the placebo group at 24 h and 48 h post-EIMD (p<0.05). As to the fatigue index, FIG. 4C showed that the HK-PS23 group had significantly lower increase at 24 h post-EIMD than the placebo group (p<0.05), and the L-PS23 and HK-PS23 groups had significantly smaller increase at 48 h post-EIMD than the placebo group (p<0.05). In addition, these results indicated significant time effects for relative mean power, relative peak power and fatigue index (p<0.0001).

By the above results, it may be found that both L-PS23 and HK-PS23 could significantly prevent the decrease in jump-related strength performance after EIMD and had better recovery effects, and could also improve the performance of Wingate's anaerobic power.

### Example 4: Effects of L-PS23 and HK-PS23 Supplementation on Biomarkers of Muscle Damage, Inflammation, Oxidative Stress, and Anabolic Hormones

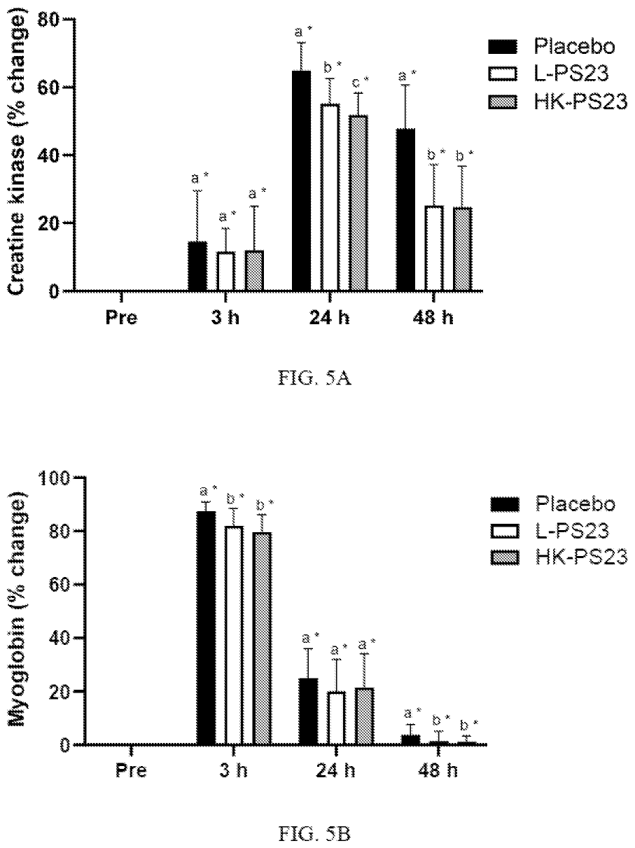

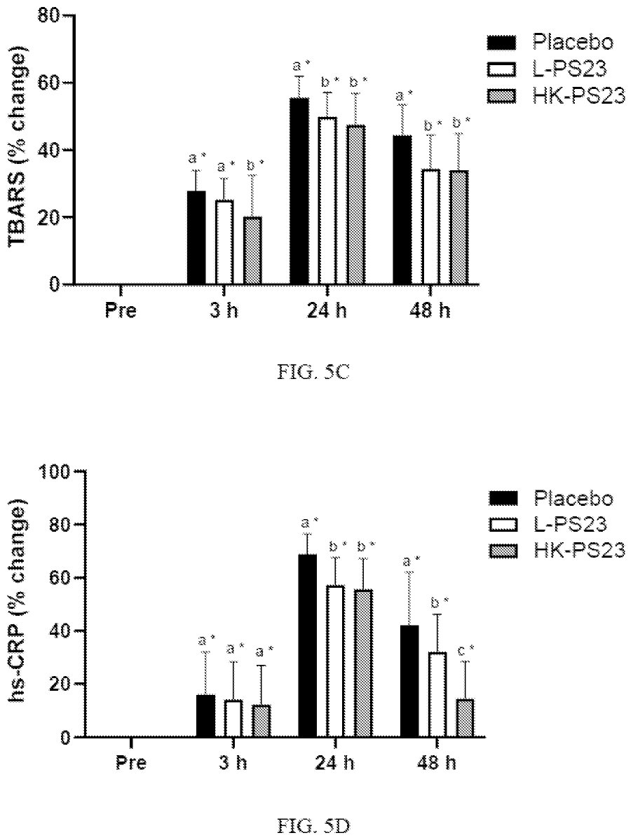

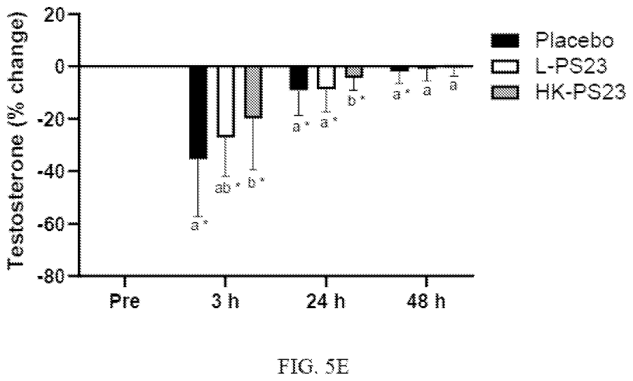

The results of biomarker assessments were shown in FIGS. 5A to 5E, and demonstrated that all groups showed significantly increased CK, myoglobin, TBARS and high-sensitivity C-reactive protein (hs-CRP) at 3 b, 24 h, and 48 h post-EIMD (p<0.05).

Furthermore, the increases in CK activity at 24 h and 48 h post-EIMD were significantly reduced in the L-PS23 and HK-PS23 groups as compared with the placebo group (p<0.05) (FIG. 5A). The myoglobin increase ratios in the L-PS23 and HK-PS23 groups at 3 h and 48 h post-EIMD were significantly lower than the placebo group (p<0.05) (FIG. 5B). As shown in FIG. 5C, the TBARS increase ratios in the L-PS23 and HK-PS23 groups at 24 h and 48 h post-EIMD were significantly lower than the placebo group (p<0.05), while at 3 h post-EIMD, the TBARS increase ratio in the HK-PS23 group was also significantly lower than the placebo and L-PS23 groups (p<0.05). The hs-CRP increase ratios in the L-PS23 and HK-PS23 groups at 24 h and 48 h post-EIMD were significantly lower than the placebo group (p<0.05), while at 48 h post-EIMD, the hs-CRP increase ratio in the HK-PS23 group was also significantly lower than the L-PS23 group (p<0.0001) (FIG. 5D).

Further, as shown in FIG. 5E, at 3 h and 24 h post-EIMD, the percentage changes of testosterone were significantly lower in the placebo, L-PS23, and HK-PS23 groups as compared with pre-EIMD (p<0.0001). Also, at 48 h post-EIMD, the percentage change of testosterone in the placebo group remained significantly lower than pre-EIMD, while the percentage changes of testosterone in the L-PS23 and HK-PS23 groups had recovered with no significant difference from pre-EIMD. In addition, the HK-PS23 group had significantly less loss and faster recovery of the testosterone concentration than the placebo group at 3 h and 24 h post-EIMD (p<0.05), and also significantly less loss than those of the L-PS23 group at 24 h post-EIMD (p<0.05).

These results showed that the supplementation with L-PS23 or HK-PS23 not only slowed the degree of post-exercise CK or myoglobin elevation, but also reduced oxidative stress and inflammation after EIMD. Also, the supplementation with L-PS23 or HK-PS23 had significantly reduced testosterone loss and facilitated faster recovery.

From the above, six consecutive weeks of L-PS23 or HK-PS23 supplementation significantly reduced muscle force loss and the amount of blood biomarkers of muscle damage, inflammation, and oxidative stress after EIMD, and was beneficial for accelerated recovery, suggesting the protective, recovery and improvement benefits of L. paracasei PS23 supplementation after muscle damage.

Therefore, the probiotic composition comprising L. paracasei PS23, either live or heat-killed, provided in the present disclosure may improve exercise performance, such as reducing muscle strength loss, reducing the amount of biomarkers of muscle damage, inflammation, and oxidative stress in serum, and accelerating muscle damage repair, strength, and fatigue recovery. Also, the probiotic composition provided in the present disclosure may prevent or minimize EIMD.

In addition, since the heat-killed probiotics (i.e., HK-PS23) also have the superior effect of improving exercise performance, the probiotic composition comprising HK-PS23 provided in the present disclosure is safer with longer shelf-life and is easier to transport and store.

It is obvious to a person skilled in the art that with the advancement of technology, the basic idea may be implemented in various ways. The embodiments are thus not limited to the examples described above; instead, they may vary within the scope of the claims.

The embodiments described hereinbefore may be used in any combination with each other. Several of the embodiments may be combined together to form a further embodiment. A method disclosed herein may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages.

### REFERENCE

[1] Visconti, L. M.; Cotter, J. A.; Schick, E. E.; Daniels, N.; Viray, F. E.; Purcell, C. A.; Brotman, C. B. R.; Ruhman, K. E.; Escobar, K. A. Impact of varying doses of omega-3 supplementation on muscle damage and recovery after eccentric resistance exercise. Metabolism Open. 2021, 12:100133.
[2] Owens, D. J.; Twist, C.; Cobley, J. N.; Howatson, G.; Close, G. L. Exercise-induced muscle damage: What is it, what causes it and what are the nutritional solutions? European Journal of Sport Science. 2019, 19(1):71-85.
[3] Fatouros, I. G.; Jamurtas, A. Z. Insights into the molecular etiology of exercise-induced inflammation: opportunities for optimizing performance. Journal of Inflammation Research. 2016, 9:175-186.
[4] Salehi, M.; Mashhadi, N. S.; Esfahani, P. S.; Feizi, A.; Hadi, A.; Askari, G. The effects of curcumin supplementation on muscle damage, oxidative stress, and inflammatory markers in healthy females with moderate physical activity: a randomized, double-blind, placebo-controlled clinical trial. International Journal of Preventive Medicine. 2021, 12:94.
[5] Paulsen, G.; Mikkelsen, U. R.; Raastad, T.; Peake, J. M. Leucocytes, cytokines and satellite cells: what role do they play in muscle damage and regeneration following eccentric exercise? Exercise Immunology Review. 2012, 18:42-97.
[6] Peake, J. M.; Neubauer, O.; Della Gatta, P. A.; Nosaka, K. Muscle damage and inflammation during recovery from exercise. Journal of Applied Physiology (1985). 2017, 122(3):559-570.
[7] Marián, V.; Katarína, L.; Dávid, O.; Matúš, K.; Simon, W. Improved maximum strength, vertical jump and sprint performance after 8 weeks of jump squat training with individualized loads. Journal of Sports Science and Medicine. 2016, 15(3):492-500.
[8] Hong, W. H.; Lo, S. F.; Wu, H. C.; Chiu, M. C. Effects of compression garment on muscular efficacy, proprioception, and recovery after exercise-induced muscle fatigue onset for people who exercise regularly. PLoS One. 2022, 17(2):e0264569.

What is claimed is:

1. A method for preventing or minimizing exercise-induced muscle damage (EIMD) in a subject in need thereof, comprising administering to the subject a composition comprising an effective amount of a lactic acid bacterium and a carrier thereof, wherein the lactic acid bacterium is Lactobacillus paracasei PS23 deposited under DSMZ Accession No. 32322.

2. The method according to claim 1, wherein preventing or minimizing EIMD comprises at least one of enhancing muscle strength, increasing muscle endurance, delaying or reducing muscle fatigue, accelerating muscle recovery from EIMD, and preventing or attenuating delayed onset muscle soreness (DOMS).

3. The method according to claim 1, wherein the preventing or minimizing EIMD further comprises enhancing physical capacity in the subject.

4. The method according to claim 1, wherein the preventing or minimizing EIMD further comprises improving exercise performance in the subject.

5. The method according to claim 1, wherein the preventing or minimizing EIMD further comprises reducing inflammation in the subject.

6. The method according to claim 1, wherein the preventing or minimizing EIMD further comprises reducing oxidation stress in the subject.

7. The method according to claim 1, wherein the composition is administered to the subject for modulating a serum level of at least one biomarker associated with muscle damage, oxidative stress, inflammation, or anabolic hormones in the subject.

8. The method according to claim 7, wherein after the administration, an increase of at least one of creatine kinase, myoglobin, thiobarbituric acid reactive substances, and high-sensitivity c-reactive protein resulted from EIMD is suppressed in the subject.

9. The method according to claim 7, wherein after the administration, a reduction of testosterone resulted from EIMD is suppressed in the subject.

10. The method according to claim 1, wherein the lactic acid bacterium is in a form selected from the group consisting of a live strain, a heat-killed strain, a culture, a lysate, an extract, and any combination thereof.

11. The method according to claim 1, wherein the composition is administered to the subject at an amount of at least 1×10⁶ colony-forming unit (CFU) of the lactic acid bacterium per day.

12. The method according to claim 1, wherein the lactic acid bacterium serves as a sole active ingredient in the composition for preventing or minimizing EIMD.

13. The method according to claim 1, wherein the composition is a nutritional composition, a food product, or a pharmaceutical composition.

14. The method according to claim 1, wherein the composition is in a form suitable for oral administration.

15. The method according to claim 1, wherein the composition is in a form of a concentrate, a paste, a powder, a suspension, a granule, a tablet, a pill, a capsule, a drink, or a dairy product.

16. The method according to claim 1, wherein the composition is administered to the subject with a frequency selected from the group consisting of once per week, 2 times per week, 3 times per week, 4 times per week, once every three days, 2 times every three days, once every two days, once per day, 2 times per day, 3 times per day, and 4 times per day.

17. The method according to claim 1, wherein the composition is administered to the subject for at least one week.
